(19) United States
(12) Patent Application Publication
(10) Pub. No.: US 2016/0153011 A1
(43) Pub. Date: Jun. 2, 2016

(54) Method for production of n-propanol and/or ethanol by fermentation of multiple substrates in a symbiotic manner

(71) Applicants: Michael Enzien, Lisle, IL (US); Robert Hickey, Okemos, MI (US); William Levinson, Naperville, IL (US); Walter Vermeiren, Houthalen-Helchteren (BE)

(72) Inventors: Michael Enzien, Lisle, IL (US); Robert Hickey, Okemos, MI (US); William Levinson, Naperville, IL (US); Walter Vermeiren, Houthalen-Helchteren (BE)

(21) Appl. No.: 14/958,159

(22) Filed: Dec. 3, 2015

Related U.S. Application Data
(63) Continuation of application No. 13/802,930, filed on Mar. 14, 2013, now abandoned.

(51) Int. Cl. C12P 7/14; C12P 7/04

(52) U.S. Cl. CPC C12P 7/14; C12P 7/04

(57) ABSTRACT

This invention provides methods and systems for the production of n-propanol and ethanol. Specifically, the methods and systems of the present invention use symbiotic co-cultures for the production of propanol from syngas.

### FIELD OF THE INVENTION

The invention provides methods and systems for production of short-chain alcohols, in particular n-propanol, ethanol and other C3 and C2-containing products from syngas using symbiotic co-cultures of anaerobic microorganisms.

### BACKGROUND OF THE INVENTION

Propanol is a solvent used industrially, but more importantly, it can be readily dehydrated to produce propylene which is the second largest chemical commodity in the world with production of >70 million tons/per year. Currently propylene is produced mainly by steam-cracking of naphtha or liquid petroleum gas or fluid catalytic cracking of gasoils in very large installations as a secondary product. The steam-cracking is a process that makes majorly ethylene and many other co-products, such as butylenes, butadiene and pyrolysis gasoline, all of which need to be purified and to be utilized simultaneously. Other ways to make propylene is in a refinery FCC (fluid catalytic cracking) where propylene is a byproduct from heavy gasoil cracking in proportions between 3 and 15 wt %. Propylene can also be produced by catalytic dehydrogenation of propane. Still another way to make propylene is via metathesis of butenes with ethylene.

For many centuries, simple sugars are fermented into ethanol with the help of saccharomyces cerevisae. The last decade's new routes starting from cellulose and hemicelluloses have been developed to ferment more complex carbohydrates into ethanol. Hereto, the carbohydrates need to be unlocked from the lignocellulosic biomass. Biomass consists approximately of 30% cellulose, 35% hemicelluloses and 25% lignin. The lignin fraction cannot be valorised as ethanol, as of its aromatic nature but can only be used as an energy source which is present in many cases in excess for running an industrial plant.

Several microorganisms are able to use one-carbon compounds as a carbon source and some even as energy source. Carbon dioxide is an important carbon source for phototrophs, sulfate reducers, methanogens, acetogens and chemolithotrophic microorganisms. There are essentially four systems to fix CO₂: (1) the Calvin cycle [CO₂ fixing enzyme: ribulose-1,5-bisphosphate carboxylase], (2) the reductive citric acid cycle [CO₂ fixing enzymes: 2-oxoglutarate synthase, isocitrate dehydrogenase, pyruvate synthase], (3) the acetyl-CoA pathway [CO₂ fixing enzyme: acetyl-CoA synthase, linked to CO-dehydrogenase] and (4) the 3-hydroxypropionate cycle [CO₂ fixing enzyme: acetyl-CoA carboxylase, propionyl-CoA carboxylase] (“Structural and functional relationships in Prokaryotes”, L. Barton, Springer 2005; “Carbon monoxide-dependent energy metabolism in anaerobic bacteria and archaea”, E. Oelgeschelager, M. Rother, Arch. Microbiol., 190, p. 257, 2008; “Life with carbon monoxide”, S. Ragsdale, Critical Reviews in Biochem. and Mol. Biology, 39, p. 165, 2004). Several microorganisms can also use carbonmonoxide:

Bacteria:
Acetogens (like Acetobacterium woodii, Clostridium pasteurianum etc)
Carboxydotrophs (like Alcaligenes carboxydus, Bacillus schlegelii, Pseudomonas carboxydoflava, Pseudomonas compransori)
Methanotrophs (like Pseudomonas methanica, Methylosinus methanica, Methylococcus capsulatus)
Nitrogen fixers (like Azomonas B1, Azospirillum lipoferum, Bradyrhizobium japonicum)
Phototrophs (like Rhodocyclus gelatinosa, Rhodospirillum rubrum, Spirulina platensis)
Sulfate reducers (like Desulfobacterium autotrophicum, Desulfotomaculum acetoxidans, Desulfovibrio desulfuricans, Desulfovibrio vulgaris)

Archaea:
Methanogens (like Methanobacterium, thermoautotrophicum, Methanosarcina barkeri, Methanothrix soehngenii)

Carboxydotrophs oxidize CO into CO₂ using a molybdenum-containing CO-dehydrogenase and use further the Calvin cycle to fix CO₂. Acetogens can interconvert CO—CO₂ using a Nickel-iron-containing CO-dehydrogenase. This CO-dehydrogenase is linked to an Acetyl-CoA synthase that fixes CO₂ in the Wood-Ljungdahl pathway.

Recently, more efficient routes that produce synthesis gas from carbon-containing materials and that subsequently is fermented into ethanol are being developed (“Bioconversion of synthesis gas into liquid or gaseous fuels”, K. Klasson, M. Ackerson, E. Clausen, J. Gaddy, Enzyme and Microbial Technology, 14(8), p. 602, 1992; “Fermentation of Biomass-Generated Producer Gas to Ethanol”, R. Datar, R. Shenkman, B. Cateni, R. Huhnke, R. Lewis, Biotechnology and Bioengineering, 86 (5), p. 587, 2004; “Microbiology of synthesis gas fermentation for biofuel production”, A. Hemstra, J. Sipma, A. Rinzema, A. Stams, Current Opinion in Biotechnology, 18, p. 200, 2007; “Old Acetogens, New Light”, H. Drake, A. Göβner, S. Daniel, Ann. N.Y. Acad. Sci. 1125: 100-128, 2008). Synthesis gas can be produced by gasification of the whole biomass without need to unlock certain fractions. Synthesis gas can also be produced from other feedstock via gasification: (i) coal, (ii) municipal waste (iii) plastic waste, (iv) petcoke and (v) liquid residues from refineries or from the paper industry (black liquor). Synthesis gas can also be produced from natural gas via steam-reforming or autothermal reforming (partial oxidation).

The biochemical pathway of synthesis gas conversion is described by the Wood-Ljungdahl Pathway. Fermentation of syngas offers several advantages such as high specificity of biocatalysts, lower energy costs (because of low pressure and low temperature bioconversion conditions), greater resistance to biocatalyst poisoning and nearly no constraint for a preset H₂ to CO ratio (“Reactor design issues for synthesis-gas fermentations” M. Bredwell, P. Srivastava, R. Worden, Biotechnology Progress 15, 834-844, 1999; “Biological conversion of synthesis gas into fuels”, K. Klasson, C. Ackerson, E. Clausen, J. Gaddy, International Journal of Hydrogen Energy 17, p. 281, 1992). Acetogens are a group of anaerobic bacteria able to convert syngas components, like CO, CO₂ and H₂ to acetate via the reductive acetyl-CoA or the Wood-Ljungdahl pathway.

Several anaerobic bacteria have been isolated that have the ability to ferment syngas to ethanol, acetic acid and other useful end products. Clostridium ljungdahlii and Clostridium autoethanogenum, were two of the first known organisms to convert CO, CO₂ and H₂ to ethanol and acetic acid. Commonly known as acetogens, these microorganisms have the ability to reduce CO2 to acetate in order to produce required energy and to produce cell mass. The overall stoichiometry for the synthesis of ethanol using three different combinations of syngas components is as follows (J. Vega, S. Prieto, B. Elmore, E. Clausen, J. Gaddy, “The Biological Production of Ethanol from Synthesis Gas”, Applied Biochemistry and Biotechnology, 20-1, p. 781, 1989):

6CO+3H₂O→CH₃CH₂OH+4CO₂

2CO₂+6H₂→CH₃CH₂OH+3H₂O

6CO+6H₂→2CH₃CH₂OH+2CO₂

Acetogenic bacteria are obligate anaerobic bacteria that utilize the reductive acetyl-CoA pathway as their predominant mechanism for the reductive synthesis of acetyl-CoA from CO₂ (Drake, H. L. (1994). Acetogenesis. New York: Chapman & Hall). This group of microorganisms is even more versatile in the sense that they can use simple gases like CO₂/H₂ and CO as well as sugars, carboxylic acids, alcohols and amino acids (i) as terminal electron-accepting, energy-conserving process, and (ii) as mechanism for the synthesis of cell carbon from CO₂″ (Drake, H. L. (1994). Acetogenesis. New York: Chapman & Hall). Like other anaerobes, acetogens require a terminal electron acceptor different from oxygen. In the acetyl-CoA pathway, CO₂ serves as an electron acceptor and H₂ serves as the electron donor. The synthesis of acetyl-CoA from CO₂ and H₂ requires an 8-electron reduction of CO₂ involving the following three steps:

Formation of the carbonyl precursor of acetyl-CoA
Formation of the methyl precursor of acetyl-CoA
Condensation of the above two precursors to form acetyl-CoA.

Clostridium ljungdahlii, one of the first autotrophic microorganisms known to ferment synthesis gas to ethanol, was isolated in 1987, and, as an acetogen, favours the production of acetate during its active growth phase (acetogenesis) while ethanol is produced primarily as a non-growth-related product (solventogenesis) (“Biological conversion of synthesis gas into fuels”, K. Klasson, C. Ackerson, E. Clausen, J. Gaddy, International Journal of Hydrogen Energy 17, p. 281, 1992). In its solventogensis stage wherein ethanol is primarily produced from syngas, Clostidiucm ljungdahlii acts as a homoacetogen.

Clostridium autoethanogenum is a strictly anaerobic, gram-positive, spore-forming, rod-like, motile bacterium which metabolizes CO to form ethanol, acetate and CO₂ as end products, beside it ability to use CO₂ and H₂, pyruvate, xylose, arabinose, fructose, rhamnose and L-glutamate as substrates (J. Abrini, H. Naveau, E. Nyns,), “Clostridium autoethanogenum, Sp-Nov, an Anaerobic Bacterium That Produces Ethanol from Carbon-Monoxide”, Archives of Microbiology, 161(4), p. 345, 1994). With syngas as a substrate, Clostridium autoethanogenum also acts as homoacetogen and primarily produces ethanol when in solventogenisis.

Clostridium carboxidivorans P7 is a solvent-producing anaerobe, which was isolated from the sediment of an agricultural settling lagoon. It is motile, gram-positive, spore-forming and primarily acetogenic, forming acetate, ethanol, butyrate, and butanol as end-products. (J. Liou, D. Balkwill, G. Drake, R. Tanner, “Clostridium carboxidivorans sp. nov., a solvent-producing clostridium isolated from an agricultural settling lagoon, and reclassification of the acetogen Clostridium scatologenes strain SL1 as Clostridium drakei sp. nov.”, International Journal of Systematic and Evolutionary Microbiology, 55(5), p. 2085, 2005). Clostridium carboxidivorans will typically produce both ethanol and butanol from syngas.

Anaerobic acetogenic microorganisms offer a viable route to convert syngas, such as waste gases in combination with carbohydrates or proteins, to useful products, such as ethanol and n-propanol, via an indirect fermentation process. Such bacteria catalyze the conversion of H₂ and CO₂ and/or CO to acids and/or alcohols with higher specificity, higher yields and lower energy costs than can be attained by traditional production processes. While many of the anaerobic microorganisms utilized in the fermentation of ethanol also produce a small amount of n-propanol as a by-product, to date, no single anaerobic microorganism has been described that can utilize the direct fermentation process of syngas to produce high yields of n-propanol and ethanol.

Therefore a need in the art remains for methods using microorganisms in the production of n-propanol and ethanol using indirect fermentation.

### SUMMARY OF THE INVENTION

In broadest terms there has been discovered a method for producing alcohols, including at least either or both of n-propanol and ethanol comprising exposing gaseous substrates containing at least carbon monoxide, carbon dioxide and hydrogen or combinations thereof to an acetogenic (C1 fixing) microorganism in a first fermentation zone to produce n-propanol, acetate, and/or ethanol in the presence of organic carboxylate salts, in particular propionate salts and acetate salts produced from carbohydrates and/or proteins (hereinafter CP refers to carbohydrate and/or protein) substrate or from the acetate and/or ethanol substrate produced by the acetogenic microorganism. The organic carboxylate salts, in particular propionate salts and acetate salts are produced either in a second fermentation zone and transferred into the first fermentation zone or by feeding at least one of the CP into the first fermentation zone that also contains the C3 producing microorganism in a symbiotic relationship with the acetogenic microorganisms. In preferred aspects of this invention the acetogen is a homoacetogen.

In a more limited form of the invention, the first fermentation zone may contain together with the acetogenic microorganism, a C3-producing microorganism to provide a symbiotic co-culture of the microorganisms that increases the conversion of the gaseous substrate and CP into n-propanol or/and into propionic acid. In most cases the gaseous substrate is syngas and the C3-producing microorganism is a propionogen.

In a more limited form, there has been discovered an anaerobic symbiotic system for conversion of syngas and CP to n-propanol or/and to propionic acid, the system comprising syngas, culture media, a C1-fixing microorganism and a C3-producing microorganism in one or more bioreactors. Usually, in this form of the invention the C3-producing microorganism is again a propionogen.

### BRIEF DESCRIPTION OF THE DRAWINGS

These and other objects, features, and embodiments of the invention will be better understood from the following detailed description taken in conjunction with the drawings, wherein (the word carboxylate means in general terms all organic compounds having at least one carboxyl-moiety, beside eventually other functional groups like alcohols, double bonds, ketons etc.):

FIG. 1 is a schematic diagram of microorganisms, feed substrates and products. The symbiotic C3 fixing microorganism converts CP to organic carboxylates, including at least propionate and acetate and converts (secondarily) H₂/CO/CO₂ to C3-containing products, namely propionate and n-propanol. Other organic carboxylates can be succinate and lactate. The C1-fixing microorganism also converts the carboxylates into their corresponding alcohols, including at least propionate to n-propanol, which becomes the primary end product.

FIG. 2 is a schematic diagram of an embodiment showing a vessel for the production of organic carboxylates, including at least propionate and acetate that receives the CP feed and showing another fermentation vessel that receives the carboxylates, including at least propionate and acetate salts, receives a gas substrate of carbon monoxide, carbon dioxide and hydrogen, and contains the acetogenic microorganisms, producing the corresponding alcohols, including at least n-propanol and ethanol.

FIG. 3 is a schematic diagram of an embodiment showing a single vessel for the production of alcohols, including at least n-propanol and/or ethanol. The vessel receives the CP feed; receives the gas substrate of carbon monoxide, carbon dioxide and hydrogen; contains, as a co-culture, the acetogenic microorganism and the C₃-producing microorganisms, which results in the formation of the organic carboxylates, including at least propionate and acetate salts that are converted by the acetogenic microorganisms into the corresponding alcohols, n-propanol and ethanol.

The C1-fixing microorganism produces ethanol and acetate from syngas. The C3-producing microorganism converts the ethanol, acetate and (secondarily) H₂/CO/CO₂ to C3-containing products, namely propionate and n-propanol. The C3-producing microorganism converts the CP into mixed carboxylates, including at least propionate and/or acetate, but can also include other carboxylates like succinate and lactate. The C1-fixing microorganism also converts the carboxylates into the corresponding alcohols, including at least propionate to n-propanol, which becomes, along with any ethanol, the primary end product.

### DETAILED DESCRIPTION OF THE INVENTION

The invention provides methods for the production of propanol and other C3-containing products from syngas by the use of symbiotic co-cultures of anaerobic microorganisms. In other aspects, the invention provides anaerobic systems for conversion of syngas to n-propanol.

As used herein, synthesis gas (syngas) is a gas containing carbon monoxide, carbon dioxide and frequently hydrogen. “Syngas” includes streams that contain carbon dioxide in combination with hydrogen and that may include little or no carbon monoxide. “Syngas” may also include carbon monoxide gas streams that may have little or no hydrogen.

As used herein, the term “symbiotic” refers to the association of two or more different types (e.g. organisms, populations, strains, species, genera, families, etc.) of anaerobic microorganisms which are capable of forming a tightly associated metabolic symbiosis. As used herein, the term “co-culture” of microorganisms refers to joint incubation or incubation together, of the symbiotic microorganisms. In the context of the present invention, the co-culture does not require cellular population growth during the joint incubation of the symbiotic microorganisms.

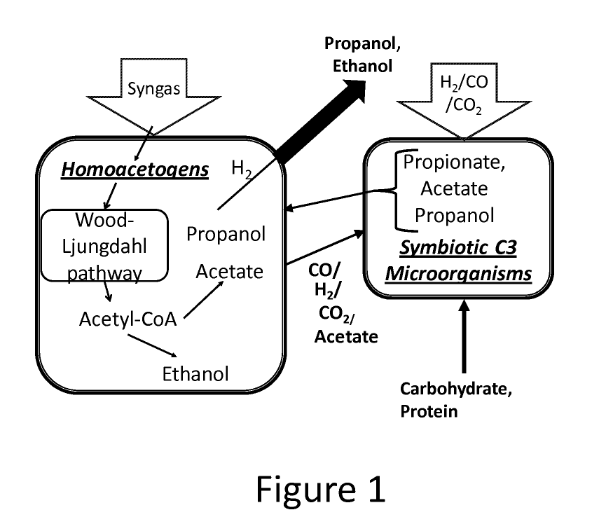

In an embodiment of the invention illustrated in FIG. 1, two types of anaerobic microorganism can be utilized to create the symbiotic relationship for production of n-propanol and/or ethanol. The first type of microorganism is one for fermenting syngas into ethanol and acetate. The second type of microorganism converts CP in a second fermentation zone to produce at least one of propionate salts and acetate salts in aqueous solution. The first type of microorganism in the symbiotic co-culture is an acetogen that serves as a primary C1-fixing microorganism and which utilizes syngas as the carbon and electron source and produces ethanol and acetate as the dissimilatory metabolite products. The second type of microorganism in the symbiotic relationship may be also capable, in addition to the CP, of growing on the dissimilatory metabolites of the acetogenic microorganisms (ethanol and acetate) as its carbon and/or electron source to produce a C3-carbon molecule, such as n-propanol or propionic acid, as its primary product, or together with syngas (as additional carbon and/or electron source) convert the metabolites of the acetogenic microorganism to C3-carbon molecules. This second microorganism shall be referred to herein as the C3 producing microorganism. Advantageously, the acetogenic (C1-fixing microorganism) may also be capable of converting the propionate produced by the C3-producing microorganism into n-propanol.

The acetogenic (C1-fixing) microorganisms of the invention are also homoacetogens. Homoacetogens have the ability, under anaerobic conditions, to produce acetic acid and ethanol from the substrates, CO+H₂O, or H₂+CO₂ or CO+H₂+CO₂. The CO or CO₂ provides the carbon source and the H₂ or CO provides the electron source for the reactions producing acetic acid and ethanol. The primary product produced by the fermentation of CO and/or H₂ and CO₂ by homoacetogens is ethanol according to the following reactions:

6CO+3H₂O→C₂H₅OH+4CO₂

6H₂+2CO₂→C₂H₅OH+3H₂O

Homoacetogens may also produce acetate. Acetate production occurs via the following reactions:

4CO+2H₂O CH₃COOH+2CO₂

4H₂+2CO₂→CH₃COOH+2H₂O

C1-fixing microorganisms suitable for use in the inventive method include, without limitation, homoacetogens such as Clostridium ljungdahlii, Clostridium autoethanogenum, Clostridium ragsdalei, and Clostridium coskatii. Additional C1-fixing microorganisms that are suitable for the invention include Alkalibaculum bacchi, Clostridium thermoaceticum, and Clostridium aceticum.

Pathways for the production of oxygenates having three carbons: Propionic acid production: Propionibacterium species (Propionibacterium acidipropionici, Propionibacterium acnes, Propionibacterium cyclohexanicum, Propionibacterium freudenreichii, Propionibacterium freudenreichii shermanii, Propionibacterium pentosaecum) and several other anaerobic bacteria such as Desulfobulbus propionicus, Pectinatus frisingensis, Pelobacter propionicus, Veillonella, Selenomonas, Fusobacterium, Bacteroides fragile, Prevotella ruminicola, Megasphaera elsdenii, Bacteroides vulgates, and Clostridium, in particular Clostridium propionicum, produce propionic acid as a main fermentation product (Playne M., “Propionic and butyric acids”, In: Moo-Young M, editor. Comprehensive biotechnology, New York: Pergamon Press, vol 3, p 731-759, 1985; Seshadri N, Mukhopadhyay S., “Influence of environmental parameters on propionic acid upstream bioprocessing by Propionibacterium acidipropionici”, J. Biotechnology 29, p. 321-328, 1993). In swiss-type cheeses, propionibacteria consume lactate and produce propionic acid, acetic acid, and CO2. In general, a broad range of substrates can be converted into propionic acid, like glucose, lactose, sucrose, xylose, glycerol and lactate. Propionibacteria are Gram-positive, non-motile, non-sporulating, short-rodshaped, mesophilic anaerobes. The genus of Propionibacterium, belonging to the class of high G+C actinobacteria is divided into two groups: the “cutaneous” and the “dairy” Propionibacteria, based on their habitat (Stackebrandt, E., Cummins, C., Johnson, J., “The Genus Propionibacterium”, in The Prokaryotes, E. Balows, H. Truper, M. Dworkin, W. Harder, K. Scheifer, eds., 2006).

Dicarboxylic Pathway:

Propionibacteria convert carbohydrates or protein to produce propionic acid as a main product via the mainly dicarboxylic acid pathway (also called the Wood-Werkman cycle, succinate, randomising or the methyl-malonyl-CoA pathway). Glycolysis pathway catabolyses glucose into phosphoenolpyruvate (PEP), an energy-rich metabolite. Two alternative glycolysis pathways exist: Embden-Meyerhorf-Parnaz (EMP) pathway and Hexose Monophosphate (HMP) pathway. In the EMP pathway, 1 mole of glucose is converted into 2 moles of PEP and 2 moles of NADH, while in the HMP pathway 1 mole of glucose provides 5/3 moles of PEP and 11/3 moles of NADH. PEP is further converted into two possible intermediates, pyruvate and oxaloacetate. The majority of PEP is converted into pyruvate whereas the remaining PEP is converted into oxaloacetate. For pyruvate production, 1 mole of PEP is converted into 1 mole of pyruvate and 1 mole of ATP is obtained from a transfer of one phosphoryl moiety from PEP to ADP. The total ATP obtained from the EMP and HMP pathways per mole of glucose is 2 and 5/3 moles, respectively. Glycolysis via the EMP pathway provides a lower amount of NADH (EMP: HMP=2: 11/3) but a higher amount of ATP (EMP: HMP=2: 5/3). The ratio of EMP to HMP pathway contribution in glycolysis is dependent on propionibacterium species, substrates and fermentation conditions. At the pyruvate node, pyruvate is directed toward three main pathways. Most of pyruvate is converted into propionic acid via the Wood-Werkman cycle. Some of pyruvate converts into acetate while some is incorporated into biomass. In the propionate formation pathway, pyruvate enters the Wood-Werkman cycle, via a transcarboxylation of a carboxyl-moiety from methylmalonyl-CoA to pyruvate, catalysed by oxaloacetate transcarboxylase in a coupled reaction of pyruvate to oxaloacetate and methylmalonyl CoA to propionyl CoA. In this coupled reaction, the carboxyl group transferred from methylmalonyl CoA to pyruvate to form propionyl CoA and oxaloacetate is never released from the reaction or no exchange between this carboxyl group with the dissolved CO₂ in the fermentation broth is observed (Wood H G., “Metabolic cycles in the fermentation of propionic acid”, in Current Topics in Cellular regulation, Estabrook and Srera R W, eds., New York: Academic Press. vol 18, p 225-287, 1981). Because of this transcarboxylation reaction, CO2 fixation is minimal and only used to produce catalytic amounts of oxaloacetate to reinitiate the cycle when for instance succinate accumulates as end-product. Under such circumstances, oxaloacetate is generated by condensation of CO2 with phosphoenolpyruvate catalysed by a PEP carboxylase. Subsequently, oxaloacetate is converted into malate by malate dehydrogenase, malate into fumarate by fumarase and further fumarate to succinate, catalyzed by succinate dehydrogenase. After that succinate is converted into succinyl-CoA, which is then converted into methylmalonyl-CoA. Methylmalonyl-CoA is converted into propionyl-CoA by oxaloacetate transcarboxylase. At the end of the cycle, propionyl-CoA is converted into propionate along with a coupled reaction of succinate to succinyl-CoA, catalysed by propionyl-CoA: succinate transferase. After 1 mole of pyruvate enters the Wood-Werkman cycle, 1 mole of propionate, 2 moles of NAD+, and 1 mole of ATP are generated. Beside propionic acid as main fermentation product, produced in the Wood-Werkman cycle, also NAD+ regeneration for glycolysis occurs in this cycle.

In the acetate branch pathway, pyruvate converts to acetyl-CoA and CO2, catalyzed by pyruvate dehydrogenase complex. Acetyl-CoA is converted into acetyl-phosphate by phosphotransacetylase and further acetyl-phosphate to acetate, catalyzed by acetate kinase. In the acetate branch pathway, 1 mole of acetate, CO2, NADH, and ATP are obtained from 1 mole of pyruvate. Propionic acid production is usually accompanied by the acetate formation as a major ATP production route supplying energy for cellular metabolism. The following equations represent a theoretical formulation of propionic acid fermentation from glucose or lactate (P. Piveteau, Lait, 79, p. 23, 1999):

1.5 glucose+6Pi+6ADP→2 propionate+acetate+CO2+2H2O+6ATP

3 lactic acid+3Pi+3ADP→2 propionate+acetate+CO2+2H2O+3ATP

According to these equations, the theoretical maximum yield from glucose is 66.7 C-mole % or 54.8 wt % of propionic acid, 22.2 C-mole % or 22 wt % of acetic acid, 11.1 C-mole % or 17 wt % of CO2. The theoretically propionic acid to acetic acid (P/A) molar ratio is 2:1. A shift in the metabolic pathway towards the production of propionic acid can be accomplished by using carbon sources with higher reductive level (shift from heterofermentative to homofermentative acid production). A higher reductive level of substrate can cause significant increase in the P/A ratio due to the intracellular NADH/NAD+ balance. A better efficiency of propionic acid production from glycerol could be expected because of its higher reduction level compared to conventional substrates. Effectively, a propionic acid yield of 84.4 C-mole % and a low acetic acid production (P/A molar ratio reaching 37) have been obtained from glycerol with P. acidipropionici (Barbirato, F., Chedaille, D. and Bories, A., “Propionic acid fermentation from glycerol: comparison with conventional substrates”, Appl Microbiol Biotechnol, 47, p. 441-446, 1997). This strain also produces somen-propanol from glycerol, indicating that when the substrate has a higher reduction level also products with a higher reduction level can be produced because of the better NADH/NAD+ balance.

Glycerol→propionate+1H₂O

Himmi et. al. compared the fermentation of glycerol and glucose and product formation for P. acidipropionici and P. freudenreichii ssp. shermanii. Fermentation end-products were propionic acid as the major product, acetic acid as the main byproduct and two minor metabolites, n-propanol and succinic acid. The yield of propionic acid was up to 79 C-mole % (64 wt %) with glycerol as the carbon source (Himmi, E. H., Bories, A., Boussaid, A. and Hassani, L., “Propionic acid fermentation of glycerol and glucose by Propionibacterium acidipropionici and Propionibacterium freudenreichii ssp. Shermanii”, Appl Microbiol Biotechnol, 53, p. 435-440, 2000). Rumen microorganisms that ferment lactate via the dicarboxylic acid pathway, produce more propionate relative to acetate when hydrogen is added (M. Schulmanda and D. Valentino, “Factors Influencing Rumen Fermentation: Effect of Hydrogen on formation of Propionate”, Journal of Dairy Science, vol. 59 (8), p. 1444-1451, 1976). Acetic acid was almost eliminated when a high H2 pressure was applied during the fermentation with Propionispira arboris containing hydrogenase (Thompson T. E, Conrad R, Zeikus J. G., “Regulation of carbon and electron flow in Propionispira arboris: Physiological function of hydrogenase and its role in homopropionate formation”, FEMS Microbiol Lett 22, p. 265-271, 1984 and U.S. Pat. No. 4,732,855).

According to the Wood-Werkman cycle, endogenous CO2 is released with acetic acid formation by Propionibacteria from glucose, lactose, or lactate fermentation (Deborde C., Boyaval P. 2000, Interactions between pyruvate and lactate metabolism in Propionibacterium freudenreichii subsp. shermanii: In vivo 13C nuclear magnetic resonance studies, Appl Environ Microbiol 66: 2012-2020). CO2 can be fixed in Propionibacteria to form oxaloactate from PEP catalyzed by PEP carboxylase and then lead to succinate generation. Based on the metabolic pathway (Wood-Werkman cycle), CO2 (HCO3−) is required to convert phosphoenolypyruvate (PEP) into oxaloacetate by the enzyme phosphoenolypyruvate carboxylase. Through several sequential reactions, oxaloacetate is finally converted to propionic acid. In case of glycerol as substrate, nearly no acetate and hence CO2 is produced. Applying an exogenous CO2 pressure during fermentation has an positive effect on metabolite production rate and in particular a higher succinate accumulation thanks to the higher PEP carboxylation activity (“Effect of carbon dioxide on propionic acid productivity from glycerol by Propionibacterium acidipropionici”, An Zhang and Shang-Tian Yang, SIM annual meeting and Exhibition, San Diego, 2008).

Most propionic acid producing bacteria have the enzymes of the tricarboxylic acid cycle (TCA) which explain the variable P/A ratios for different strains. Some of the acetyl-CoA can be utilized in the TCA cycle by condensation with pyruvate into citrate. The end result is that more CO₂ is produced in the TCA cycle through the decarboxylations and less acetate is secreted. P/A ratios from 2.1 to 14.7 and CO₂/acetate ratio from 1.0 to 6.3 have been reported from glucose (Wood H G., “Metabolic cycles in the fermentation of propionic acid”, in Current Topics in Cellular regulation, Estabrook and Srera R W, eds., New York: Academic Press. vol 18, p 225-287, 1981).

Pelobacter propionicus, using the dicarboxylic acid pathway, has been show to grow on ethanol as substrate while producing propionate in presence of CO2 (Schink, B., Kremer, D. and Hansen, T., “Pathway of propionate formation from ethanol in Pelobacter propionicus”, Arch. Microbiol. 147, 321-327, 1987 and S. Seeliger, P. Janssen, B. Schink, “Energetics and kinetics of lactate fermentation to acetate and propionate via methylmalonyl-CoA or acrylyl-CoA”, FEMS Microbiology Letters, 211, pp. 65-70, 2002). When ethanol is fed together with CO2 and hydrogen, significant amounts of n-propanol are produced. Ethanol is converted into acetyl-CoA (via acetaldehyde) while producing electrons for the carboxylation of acetyl-CoA into pyruvate, catalysed by pyruvate synthase. Combined with the dicarboxylic acid pathway propionate is produced from ethanol and CO2 (Schink et al., 1987).

3 ethanol+2HCO₃_{^{−}}→2 propionate-+acetate-+H++3H₂0

Pelobacter propionicus is not able to reductively convert acetate and CO2 into propionate whereas Desulfobulbus propionicus does make propionate from acetate and CO2 (Schink et al., 1987).

acetate-+HCO₃_{^{−}}+3H₂→propionate-+3H₂0

Acrylate pathway: Though many bacteria can ferment a variety of substrates anaerobically into lactate as end product, some can further reduce the lactate into propionate, like Clostrium propionicum, Clostrium neopropionicum, Megasphaera elsdenii and Prevotella ruminicola (P. Boyaval, C. Cone, “Production of propionic acid”, Lait, 75, 453-461, 1995) by using the acryloyl-CoA pathway. Several substrates (sugars, ethanol and some aminoacids) that can be converted into pyruvate as intermediate can be further reduced into propionate as main product with acetate and butyrate as co-product. The key reaction is the lactoyl-CoA dehydration into acryloyl-CoA that is subsequently reduced to propionyl-CoA. The electrons for this reduction are provided by the oxidation of pyruvate/lactate into acetate and CO2 (G. Gottschalk, “Bacterial Metabolism”, 2nd ed., Springer, New York, 1986).

Clostridium neopropionicum (strain X4), using the acrylate pathway, is able to convert ethanol and CO2 into acetate, propionate and some n-propanol (J. Tholozan, J. Touzel, E. Samain, J. trivet, G. Prensier and G. Albagnac, “Clostridium neopropionicum sp. Nov., a strict anaerobic bacterium fermenting ethanol to propionate through acrylate pathway”, Arch. Microbiol., 157, p. 249-257, 1992). As for the dicarboxylic acid pathway, the intermediate acetyl-CoA produced from the substrate ethanol is linked to the acrylate pathway via the pyruvate synthase that converts acetyl-CoA into pyruvate by carboxylation with CO2.

Recently, an alternative route leading to acryloyl-CoA consists in the conversion of acetyl-CoA into malonyl-CoA by carboxylation with CO2. The malonyl-CoA is further converted into acryloyl-CoA via four steps implicating malonate-semialdehyde, hydroxypropanoate, hydroxypropanoyl-CoA and finally acryloyl-CoA. Acryloyl-CoA produced by this pathway is subsequently reduced to propionyl-CoA similarly to the reactions leading to acryloyl-CoA by dehydratation of lactoyl-CoA (J. Zarzycki, “Identifying the missings steps of the autotrophic 3-hydroxypropionate CO2 fixation cycle in Chloroflexus aurantiacus, PNAS, 106(50), p. 21317, 2009; I. Berg, “A 3-hydroxypropionate/4-hydroxybutyrate autotrophic carbon dioxide assimilation pathway in archaea, Science, 318, p. 1782, 2007).

Preferably, the symbiotic C3-producing microorganisms of the invention are capable of growing on ethanol and/or acetate as their primary carbon source. These microorganisms include, but are not limited to, Pelobacter propionicus, Clostridium neopropionicum, Clostridium propionicum, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosymbioticum, Pelotomaculum thermopropionicum, and Pelotomaculum schinkii. In particular embodiments of the invention, the C3-producing microorganisms are propionogens. Propionogens refers to any microorganism capable of converting syngas intermediates, such as ethanol and acetate, to propionic acid and n-propanol. Propionogens of the invention utilize one of at least two distinct pathways for the conversion of syngas to propionate—the methylmalonyl-succinate pathway and the lactate-acrylate pathway.

The symbiotic cultures of the present invention have the capability in a spatially separated symbiotic relationship or as co-cultures to produce n-propanol and ethanol from CP sources and synthesis gas. The propionic acid producing bacteria may receive a single feed from CP sources and may optionally receive ethanol and acetate from the second fermentation zone.

Suitable CP sources consist of polyols, (like glycerol and sorbitol), carbohydrates, (like glucose, fructose, lactose, oligocarbohydrates, polycarbohydrates), hydroxyalcohols (like lactate), aminoacids, oligopeptides, polypeptides or any chemical combination of carbohydrate with aminoacids or combinations of the latter.

Substrates for the C1 fixing cultures can include “waste” gases such as syngas, oil refinery waste gases, steel manufacturing waste gases, gases produced by steam, autothermal or combined reforming of natural gas or naphtha, biogas and products of biomass, coal or refinery residue's gasification or mixtures of the latter. Sources also include gases (containing some H₂) which are produced by yeast, clostridial fermentations, and gasified cellulosic materials. Such gaseous substrates may be produced as byproducts of other processes or may be produced specifically for use in the methods of the present invention. Those of skill in the art will recognize that any source of substrate gas may be used in the practice of the present invention, so long as it is possible to provide the microorganisms of the co-culture with sufficient quantities of the substrate gases under conditions suitable for the bacterium to carry out the fermentation reactions.

In one preferred embodiment of the invention, the source of CO, CO₂ and H₂ is syngas. Syngas for use as a substrate may be obtained, for example, as a gaseous product of coal or refinery residues gasification. Syngas may also be produced by reforming natural gas or naphtha, for example by the reforming of natural gas in a steam methane reformer. Alternatively, syngas can be produced by gasification of readily available low-cost agricultural raw materials expressly for the purpose of bacterial fermentation, thereby providing a route for indirect fermentation of biomass to alcohol. There are numerous examples of raw materials which can be converted to syngas, as most types of vegetation could be used for this purpose. Suitable raw materials include, but are not limited to, perennial grasses such as switch grass, crop residues such as corn stover, processing wastes such as sawdust, byproducts from sugar cane harvesting (bagasse) or palm oil production, etc. Those of skill in the art are familiar with the generation of syngas from such starting materials. In general, syngas is generated in a gasifier from dried biomass primarily by pyrolysis, partial oxidation, and steam reforming, the primary products being CO, H₂ and CO₂. The terms “gasification” and “pyrolysis” refer to similar processes; both processes limit the amount of oxygen to which the biomass is exposed. The term “gasification” is sometimes used to include both gasification and pyrolysis.

Combinations of sources for substrate gases fed into the second fermentation process may also be utilized to alter the concentration of components in the feed stream to the bioreactor. For example, the primary source of CO, CO₂ and H₂ may be syngas, which typically exhibits a concentration ratio of 37% CO, 35% H₂, and 18% CO₂, but the syngas may be supplemented with gas from other sources to enrich the level of CO (i.e., steel mill waste gas is enriched in CO) Or H₂.

The symbiotic co-cultures, whether the CP is in a separate fermentation zone from the C1 fixing microorganism or both first and second cultures are together in the same vessel, the present invention must be cultured under anaerobic conditions.

As used herein, “anaerobic conditions” means the level of oxygen (O₂) is below 0.5 parts per million in the gas phase of the environment to which the microorganisms are exposed. One of skill in the art will be familiar with the standard anaerobic techniques for culturing these microorganisms (Balch and Wolfe, 1976, Appl. Environ. Microbiol. 32:781-791; Balch et al., 1979, Microbiol. Rev. 43:260-296).

Currently, no natural symbiotic pairings are able to produce n-propanol and/or ethanol from a CP feed with the combination of one microorganism to produce acetate and propionate from CP sources in combination with at least one carboxydotrophic microorganism to convert propionate and acetate to n-propanol and/or ethanol with both microorganisms operating under anaerobic conditions. The above types or microorganism when paired together under the correct nutrient conditions and selection pressures can be forced to form these symbiotic pairings which will produce n-propanol and/or ethanol from, in the simplest from, CP substrates.

Another method for establishing a symbiotic association capable of converting a propionate salts and acetate salts to n-propanol and/or ethanol involves the growing of two or more defined cultures and establishing the pairing of these separate cultures. A person skilled in the art would appreciate that there are numerous methods of pairing two or more defined cultures. For example, one method involves first growing a known C1-fixing carboxydotrophic microorganism in a fermenter with syngas as the only carbon and electron source. In a preferred embodiment, the carboxydotroph microorganism will produce ethanol and, at the same time, a known acetate and propionate producing culture is grown in a separate fermentor on a CP feed. The carboxydotrophic microorganism is preferably a homoacetogen. Once the carboxydotrophic microorganism has reached steady state with respect to ethanol and/or acetate productivity, a known acetate and propionate culture is seeded into the fermenter along with the appropriate CP feed stock.

Another method of pairing involves first growing the acetate and propionate producing microorganism in a fermenter until maximum productivity target of propionate and acetate has been reached. This stage of fermentation should have syngas as the sparging gas to acclimate the culture to syngas. Once the maximum productivity target has been reached a seed culture of the C1-fixing carboxydotrophic microorganism is added directly to the fermenter containing the acetate and propionate producing culture. Syngas mass transfer to the fermentation vessels is gradually increased to balance the gas consumption of the C1-fixing carboxydotrophic microorganims. A modification of this last method of establishing a symbiotic culture involves first growing the acetate and propionate producing culture in a fermenter with a biofilm support material that is either stationary or floating within the reactor. An example of such material is the Mutag Biochips. This method allows either microorganism to first establish a biofilm on the carrier material thereby increasing the cell retention time versus the hydraulic retention of the fermenter. Again, target propionate/acetate productivity is reached before seeding the fermenter with the C1-fixing homoacetogen.

The last method to establish a symbiotic culture capable of producing n-propanol and ethanol from CP and optionally syngas involves the initial mixing together of two or more cultures, one of which is a C1-fixing homoacetogen capable of growing on syngas and producing ethanol and acetate but will also convert acetate and propionate to their respective alcohols. The other culture(s) is a C3-producing bacteria capable of converting either ethanol or acetate, or CP to propionic acid.

A suitable medium composition used to grow and maintain symbiotic co-cultures or separately grown cultures used for sequential fermentations, includes a defined media formulation. The standard growth medium is made from stock solutions which result in the following final composition per Liter of medium. The amounts given are in grams unless stated otherwise. Minerals: NaCl, 2; NH₄Cl, 25; KCl, 2.5; KH₂PO₄, 2.5; MgSO₄.7H₂O, 0.5; CaCl₂.2H₂O, 0.1. Trace metals: MnSO₄.H₂O, 0.01; Fe(NH₄)₂(SO₄)₂.6H2O, 0.008; CoCl₂.6H2O, 0.002; ZnSO₄.7H2O, 0.01; NiCl₂.6H2O, 0.002; Na₂MoO₄.2H₂O, 0.0002, Na₂SeO₄, 0.001, Na₂WO₄, 0.002. Vitamins (amount, mg): Pyridoxine HCl, 0.10; thiamine HCl, 0.05, riboflavin, 0.05; calcium pantothenate, 0.05; thioctic acid, 0.05; p-aminobenzoic acid, 0.05; nicotinic acid, 0.05; vitamin B12, 0.05; mercaptoethanesulfonic acid, 0.05; biotin, 0.02; folic acid, 0.02. A reducing agent mixture is added to the medium at a final concentration (g/L) of cysteine (free base), 0.1; Na₂S.2H₂O, 0.1. Medium compositions can also be provided by yeast extract or corn steep liquor or supplemented with such liquids.

The methods of the present invention can be performed in any of several types of fermentation apparatuses that are known to those of skill in the art, with or without additional modifications, or in other styles of fermentation equipment that are currently under development. Examples include but are not limited to bubble column reactors, two stage bioreactors, trickle bed reactors, membrane reactors, packed bed reactors containing immobilized cells, etc. These apparatuses will be used to develop and maintain the C1-fixing homoacetogen and C3-producing propionogen cultures used to establish the symbiotic metabolic association. The chief requirements of such an apparatus include:
a. Axenicity;
b. Anaerobic conditions;
c. Suitable conditions for maintenance of temperature, pressure, and pH;
d. Sufficient quantities of substrates are supplied to the culture;
e. Optimum mass transfer performance to supply the gases to the fermentation medium
e. The end products of the fermentation can be readily recovered from the bacterial broth.

The fermentation reactor may be, for example, a traditional stirred tank reactor, a column fermenter with immobilized or suspended cells, a continuous flow type reactor, a high pressure reactor, a suspended cell reactor with cell recycle, and other examples previously listed. Furthermore, reactors may be arranged in a series and/or parallel reactor system which contains any of the above-mentioned reactors. For example, multiple reactors can be useful for growing cells under one set of conditions and generating n-propanol and/or ethanol with minimal growth under another set of conditions.

In general, fermentation of the symbiotic co-culture will be allowed to proceed until a desired level of n-propanol and/or ethanol is produced in the culture media. Preferably, the level of n-propanol and ethanol produced is in the range of 2 grams/liters to 125 grams/liters and most preferably in the range of 10 grams/liter to 75 grams/liter. Alternatively, production may be halted when a certain rate of production is achieved, e.g. when the rate of production of a desired product has declined due to, for example, build-up of bacterial waste products, reduction in substrate availability, feedback inhibition by products, reduction in the number of viable bacteria, or for any of several other reasons known to those of skill in the art. In addition, continuous culture techniques exist which allow the continual replenishment of fresh culture medium with concurrent removal of used medium, including any liquid products therein (i.e. the chemostat mode). Also techniques of cell recycle may be employed to control the cell density and hence the volumetric productivity of the fermenter.

The products that are produced by the microorganisms of this invention can be removed from the culture and purified by any of several methods that are known to those of skill in the art. For example, propanol can be removed by distillation at atmospheric pressure or under vacuum, by adsorption or by other membrane based separations processes such as pervaporation, vapor permeation and the like and further processed such as by chemical/catalytic dehydration to produce propylene.

This invention is more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined to provide additional guidance to the practitioner regarding the description of the invention.

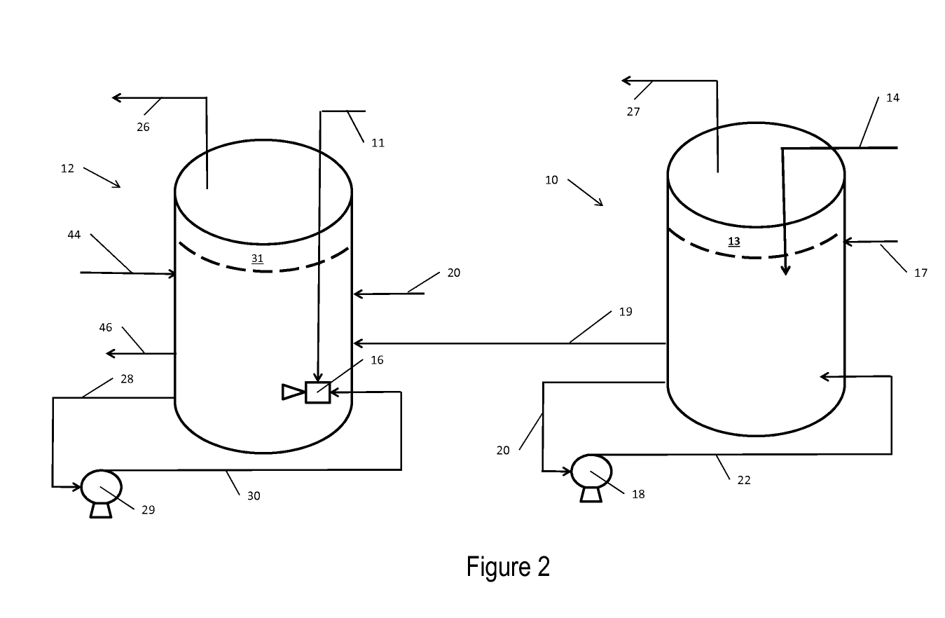

FIG. 2

In this embodiment, fermentation reactor 10, a planktonic fermentation reactor, suspends the CP converting microorganism in a liquid culture media therein and a planktonic fermentation reactor 12 suspends the propionate and acetate converting microorganism in a liquid culture medium therein. Reactor 12 is in the form of a bubble column bioreactor and reactor 10 is in the form of a continuous stirred tank reactor.

A feed comprising CP enters fermentation reactor 10 though feed line 14 for the production of propionate and acetate. The introduction of the CP feed supplies feed input for the liquid culture medium in reactor 10. A line 17 directs a portion of the liquid culture media as well nutrients into reactor 10. Reactor 10 maintains a gaseous atmosphere in top portion 13 that keeps the propionate and acetate producing microorganisms exposed to a partial pressure of 0.1 to 150 psi consisting of carbonmonoxide, carbon dioxide, hydrogen or any other inert gases or trace amounts of volatile nutrients. Additional circulation may be added to the liquid phase of reactor 10 by the pumping of liquid via line 20 pump 18 and line 22. Head space gas may be removed from reactor 10 via line 27.

A gas input line 11 supplies feed gas comprising carbon monoxide and hydrogen, and in many cases carbon dioxide to fermentation reactor 12 in combination with any returned gas. A gas injector 16 mixes the feed gas with a recirculating stream of culture media withdrawn from fermentation reactor 10 via a line 28 circulated by a pump 29 and line 30 to gas injector 16. Off-gas comprising primarily CO₂, H₂ and unreacted feed gas components exits the reactor via a line 26. The culture media of the reactor 10 containing propionate and acetate enters reactor 12 via the line 19. The gaseous atmosphere keeps the ethanol and n-propanol producing microorganism exposed to a high partial pressure of CO, CO₂ and H₂ above the liquid culture media retained by reactor 12.

A line 46 withdraws a portion of the culture media from fermentation reactor 12 for the recovery of the products such as n-propanol and, optionally, ethanol and/or acetate. The products that are produced by the microorganisms of this invention can be removed from the culture and purified by any of several methods that are known to those of skill in the art as described above in the specification. The ethanol may also be recovered by the methods described above.

The gaseous atmosphere keeps the n-propanol and ethanol producing microorganisms exposed to a high partial pressure of CO, CO₂ and H₂ while the supply of the culture media via line 19 provides propionate and/or acetate along with other nutrients via line 20 to the microorganism for the production of n-propanol and ethanol.

The culture media of the ethanol and n-propanol leaves reactor 12 via a line 46. All or a portion of the volatile alcohols are separated from the culture media, withdrawn via line 46 for recovery of propanol and ethanol from the culture media. In most cases, a line 44 will return the propionate and acetate containing media to the C1-fixing fermentation zone in fermentation reactor 12 for conversion into the corresponding alcohols.

During the reduction of the propionate and acetate in reactor 12 by means of reducing agents like CO and H2, the pH increases due to the formation of bases (without willing to be limited to any theory, the following equation demonstrates the increase in pH: CH₃CH₂COO⁻Na++2H₂→CH₃CH₂CH₂OH+Na⁺+OH⁻). Recycled liquid from the separation of the n-propanol and ethanol may contain significant quantities of propionate and/or acetate which may be returned directly to reactor 12 as part of the circulating culture media via a line 28 or 30. Recycled liquid from the separation of propanol and ethanol having an increased pH compared to the medium in reactor 10, may be completely or partially recycled to reactor 10 in order to neutralize the formation of organic carboxylates.

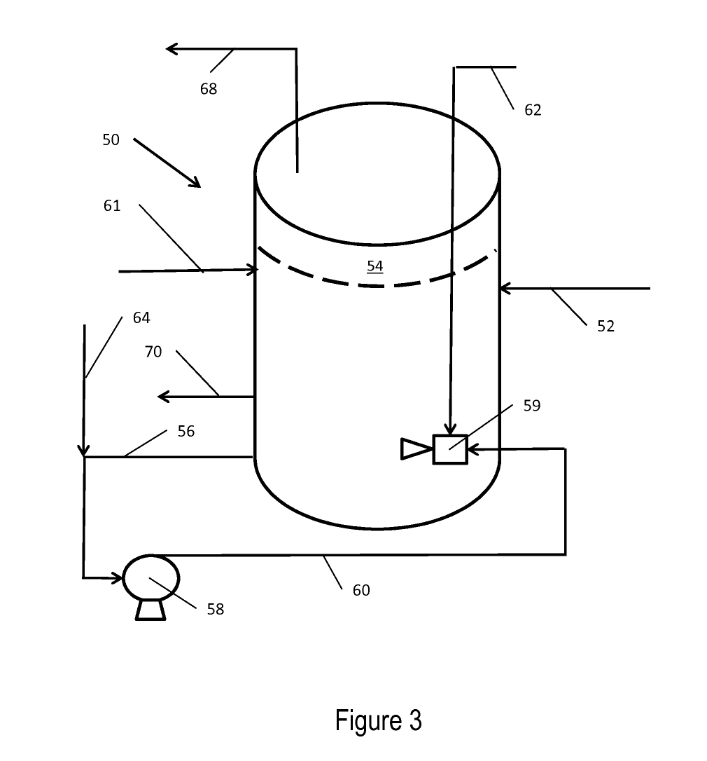

FIG. 3

In this embodiment, fermentation reactor 50, a planktonic fermentation reactor, suspends the CP converting microorganism and the acetate and propionate converting microorganism in a liquid culture media. Reactor 50 is in the form of a bubble column bioreactor.

A feed comprising CP enters fermentation reactor 50 though feed line 52 for the production of propionate and acetate. The introduction of the CP feed supplies feed input for the liquid culture medium in reactor 50. Reactor 50 maintains a gaseous atmosphere in a head space 54 that keeps the propionate and acetate producing microorganisms exposed to a partial pressure of 0.1 to 150 psi consisting of carbonmonoxide, carbon dioxide, hydrogen or any other inert gases or trace amounts of volatile nutrients. A gas input line 62 supplies feed gas comprising carbon monoxide and hydrogen, and in many cases carbon dioxide to fermentation reactor 50 in combination with any returned gas. A gas injector 59 mixes the liquid phase of reactor 50 and injects the gas as bubbles by the pumping of liquid via line 56 pump 58 and line 60. Head space gas may be removed from reactor 50 via line 68. This head space gas, also referred to as off-gas, comprising primarily CO₂, H₂ and unreacted feed gas components exits the reactor via a line 68. The culture media of the reactor 50 produces propionate and acetate in-situ. The gaseous atmosphere keeps the ethanol and n-propanol producing microorganism exposed to a high partial pressure of CO, CO₂ and H₂ above in the head space of reactor 50.

A line 70 withdraws a portion of the culture media from fermentation reactor 50 for the recovery of the products such as n-propanol and, optionally, ethanol and/or acetate. The products that are produced by the microorganisms of this invention can be removed from the culture and purified by any of several methods that are known to those of skill in the art as described above in the specification. The ethanol may also be recovered by the methods described above.

The culture media of the ethanol and n-propanol leaves reactor 50 via a line 70. If desired, all or a portion of the culture media may be withdrawn via line 70 for recovery of n-propanol and ethanol from the culture media. In most cases, a line 64 will return the propionate and acetate containing media to reactor 50 for conversion of the proprionate to n-propanol and acetate to ethanol.

Recycled liquid from the separation of the n-propanol and ethanol may contain significant quantities of propionate and/or acetate which may be returned directly to reactor 50 as part of the circulating culture media via a line 60 or separately recovered by the previously described methods.

The Examples which follow are illustrative of specific embodiments of the invention, and various uses thereof. They are set forth for explanatory purposes only, and are not to be taken as limiting the invention.

### Example 1

### C13-Labeled Propionic Acid Conversion to n-Propanol

To demonstrate that homoacetogen cultures growing on syngas convert propionic acid to n-propanol and other fermentation byproducts, C13-propionic acid experiments were performed. C13-propionic acid was fed to homoacetogen culture, Clostridium coskatti, at a concentration of 100 mM in a serum bottle with syngas in the headspace and incubated at 37° C. Samples were withdrawn from the serum bottles at 2 hrs, 24 hrs and 1 week. GC-MC was used to identify the products containing the heavy stable isotope C13. C13 products were found in the n-propanol peak and there was non-propanol produced without the C13 label. In addition there were no other products formed that contained the C13 heavy carbon isotope or its mass fragments demonstrating that homoacetogens can reduce propionic acid to n-propanol and no other end products when growing on syngas.

### Example 2

### Propionic Acid to n-Propanol in Homoacetogen Fermenters

An ethanol producing homoacetogen fermenter, fed with bubbling syngas was continuously fed propionic acid while maintaining the pH of the fermentation broth controlled at 5.0. to investigate the rate and yield of n-propanol. The initial concentration of ethanol in the fermenter was 500 mmol/L before propionic acid feed was started. Concentrations of n-propanol reached 167 mmol/L in the fermenter when dosed 200 mmol/L/hour propionic acid. Residual propionic acid in the fermenter was 27 mmol/L; therefore the conversion efficiency to n-propanol was 83%. The concentration of ethanol in the fermenter steadily decreased as the concentration of n-propanol increased. At 167 mmol/L n-propanol the fermenter contained 250 mmol/L of ethanol. This ratio of alcohols demonstrates an electron balance based on the gas consumption rates of syngas in the fermenter. A production rate of n-propanol at steady state of 0.22 g/L/hr was achieved in the fermenter. The results show both high conversion efficiency and rates of propionic acid to n-propanol by homoacetogenic microorganisms growing on syngas. In addition, these results also showed no impact on syngas consumption with n-propanol concentrations as high as 10 g/L (167 mmol/L). These results demonstrate that in a co-fermentation with the homoacetogen partner such as C. coskatii propionic acid is readily converted to n-propanol and the residual acetic acid is recycled and converted to n-propanol by this symbiotic co culture.

1. A method for the production of alcohols, including at least one of ethanol and n-propanol comprising:
a. feeding an exogenous stream of carboxylate salts containing at least one of propionate salts and acetate salts and feeding a gaseous mixture containing carbon monoxide and hydrogen to a first fermentation zone containing acetogenic microorganisms;
b. fermenting at least one of polyols, carbohydrates and proteins in a second fermentation zone containing a second fermentation zone to produce the exogenous stream of carboxylate salts containing at least one of propionate salts and acetate salts in an aqueous solution;
c. producing acetate and ethanol from the carbon monoxide, carbon dioxide and hydrogen in the first fermentation zone by contact with the acetogenic microorganisms;
d. simultaneously with step c, reducing carboxylates containing at least acetate and propionate to produce the corresponding alcohol containing at least one of n-propanol or ethanol by contact with the acetogenic microorganisms; and,
e. recovering the corresponding alcohol containing at least one of n-propanol or ethanol from a fermentation broth recovered from the first fermentation zone.

2. The method of claim 1 wherein: a portion of the fermentation broth from the first fermentation zone is removed from a bioreactor containing the first fermentation zone, the microorganisms and carboxylates containing at least one of acetate and propionate are separated from the fermentation broth to produce an alcohol stream containing at least one of ethanol and n-propanol in an aqueous solution; at least one of acetate, propionate and inorganic salts and bases thereof are recovered as a recycle stream; and, at least part of the recycle stream is recycled to at least one of the first fermentation zone and the second fermentation zone.

3. The method of claim 2 wherein a portion of the recycle stream is recycled to the first fermentation zone to neutralize the fermentation broth in the first fermentation zone.

4. The method of claim 1 wherein the at least one of polyols, carbohydrates and proteins in the second fermentation zone are fermented into carboxylates containing at least one of acetate, propionate, succinate, butyrate and lactate.

5. The method of claim 1 wherein the at least one of acetate, propionate, succinate, butyrate and lactate are reduced in the first fermentation zone into at least one of ethanol, propanol, 1,4-butandiol and 1,2-propanediol.

6. The method of claim 1 wherein the gaseous mixture includes carbon dioxide and optionally methane.

7. The method of claim 1 wherein the first fermentation broth produces an exogenous stream of propionate salts in an aqueous solution.

8. The method of claim 1 wherein the acetogenic microorganisms consist of homoacetogenic microorganisms.

9. The method of claim 1 wherein the second fermentation broth contains a co-culture of microorganisms comprising the acetogen and a propionogen.

10. A method for the production of alcohols, containing at least one of ethanol and n-propanol comprising the following steps:
a. fermenting at least one of polyols, carbohydrates and proteins in a fermentation broth contained within a bioreactor to produce carboxylates, containing at least one of propionate salts and acetate salts;
b. feeding a gaseous mixture containing carbon monoxide, carbon dioxide and hydrogen to the bioreactor, wherein the fermentation broth contains at least one species of acetogenic microorganisms having the Acetyl-CoA pathway;
c. producing acetate and ethanol from the carbon monoxide, carbon dioxide and hydrogen by contact with the acetogenic microorganisms;
d. simultaneously with step c, reducing the carboxylates, containing at least propionate and/or acetate into the corresponding alcohols, containing at least n-propanol and/or ethanol by the at least one acetogenic microorganisms having the Acetyl-CoA pathway;
e. recovering at least one of n-propanol and ethanol from the fermentation broth.

11. The method of claim 10 wherein: the microorganisms are removed from the fermentation broth to obtain an aqueous solution of products comprising carboxylates containing at least acetate salts and/or propionate salts, acetate and/or propionate, and comprising alcohols, containing at least ethanol and/or n-propanol; the ethanol and/or n-propanol are removed at least in part from the aqueous solution; and the remaining aqueous solution, containing at least acetate and/or propionate and inorganic salts or bases are at least partially recycled back to the bioreactor.

12. The method of claim 10 wherein the at least one of polyols, carbohydrates and proteins in the bioreactor are fermented into carboxylates containing at least one of acetate, propionate, succinate, butyrate and lactate.

13. The method of claim 10 wherein the at least one of acetate, propionate, succinate, butyrate and lactate are reduced in the bioreactor into at least one of ethanol, propanol, 1,4-butandiol and 1,2-propanediol.

14. The method of claim 10 wherein the gaseous mixture includes carbon dioxide and optionally methane.

15. The method of claim 10 wherein the acetogenic microorganisms consist of homoacetogenic microorganisms.

16. The method of claim 10 wherein the fermentation broth contains a co-culture of microorganisms comprising the homoacetogen acetogen and a propionogen.
